SEQUENCE LISTING (6 sequences)

SEQ ID NO: 1 - DNA, 459 bp, Unknown
  Microorganism like bacteria or yeast
atgcatcatcatcatcatcatatgctgtatgaaagcgtgagcggcgaaattaaaagcacctttagctggctgatgctgcatcagcagcgcaaccagcatgcgcgcctgattccggtggaactgtatatgagcgataaaatgcagatttttgtgaaaaccctgaccggcaaaaccattaccctggaagtggaaagcagcgataccattgataacgtgaaaagcaaaattcaggataaagaaggcattccgccggatcagcagcgcctgatttttgcgggcaaacagctggaagatggccgcaccctgagcgattataacattcagaaagaaagcaccctgcatctggtgctgcgcctgcgcggcggccatgcggaaggcacctttaccagcgatgtgagcagctatctggaaggccaggcggcgaaagaatttattgcgtggctggtgcgcggccgcggc

SEQ ID NO: 2 - DNA, 387 bp, Unknown
  Microogranism like bacteria or yeast
atgcatcatcatcatcatcatatgctgtatgaaagcgtgagcggcgaaattaaaagcacctttagctggctgatgctgcataaaaccctgaccggcaaaaccattaccctggaagtggaaagcagcgataccattgataacgtgaaaagcaaaattcaggataaagaaggcattccgccggatcagcagcgcctgatttttgcgggcaaacagctggaagatggccgcaccctgagcgattataacattcagaaagaaagcaccctgcatctggtgctgcgcctgcgcggcggccatgcggaaggcacctttaccagcgatgtgagcagctatctggaaggccaggcggcgaaagaatttattgcgtggctggtgcgcggccgcggc

SEQ ID NO: 3 - DNA, 387 bp, Unknown
  Microorganism like bacteria or yeast
atgcatcatcatcatcatcatatgctgtatgaaagcgtgagcggcgaaattcgccgcaaaaaacgccgcaaacgcaaacgcaaaaccctgaccggcaaaaccattaccctggaagtggaaagcagcgataccattgataacgtgaaaagcaaaattcaggataaagaaggcattccgccggatcagcagcgcctgatttttgcgggcaaacagctggaagatggccgcaccctgagcgattataacattcagaaagaaagcaccctgcatctggtgctgcgcctgcgcggcggccatgcggaaggcacctttaccagcgatgtgagcagctatctggaaggccaggcggcgaaagaatttattgcgtggctggtgcgcggccgcggc

SEQ ID NO: 4 - DNA, 306 bp, Unknown
  Microorganism like bacteria or yeast
atgcatcatcatcatcatcatatgctgtatgaaagcgtgagcggcgaaattaaaagcacctttagctggctgatgctgcatcagcagcgcaaccagcatgcgcgcctgattccggtggaactgtatatgagcgataaaatgcagatttttgtgaaaaccctgaccggcaaaaccattaccctggaagtggaaagcagcgatgatgatgataaacatgcggaaggcacctttaccagcgatgtgagcagctatctggaaggccaggcggcgaaagaatttattgcgtggctggtgcgcggccgcggc

SEQ ID NO: 5 - DNA, 189 bp, Unknown
  Microorgansim like bacteria or yeast
atgcatcatcatcatcatcatatgctgtatgaaagcgtgagcggcgaaattaaaagcacctttagctggctgatgctgcatgatgatgatgataaacatgcggaaggcacctttaccagcgatgtgagcagctatctggaaggccaggcggcgaaagaatttattgcgtggctggtgcgcggccgcggc

SEQ ID NO: 6 - DNA, 189 bp, Unknown
  Microorganism like bacteria or yeast
atgcatcatcatcatcatcatatgctgtatgaaagcgtgagcggcgaaattcgccgcaaaaaacgccgcaaacgcaaacgcgatgatgatgataaacatgcggaaggcacctttaccagcgatgtgagcagctatctggaaggccaggcggcgaaagaatttattgcgtggctggtgcgcggccgcggc

(19) United States
(12) Patent Application Publication
(10) Pub. No.: US 2022/0251621 A1
(43) Pub. Date: Aug. 11, 2022

(54) PROCESS FOR PRODUCTION OF SOLUBLE RECOMBINANT PEPTIDES

(71) Applicant: SAJJALA BIO LABS PRIVATE LIMITED, Hyderabad (IN)

(72) Inventors: Bhargava Sajjala, Hyderabad (IN); Srinivasulu Yeruva, Hyderabad (IN); Suvarna Lakshmi Chagamreddy, Hyderabad (IN); Spandana Thati, Hyderabad (IN)

(21) Appl. No.: 17/728,754

(22) Filed: Apr. 25, 2022

Related U.S. Application Data
(63) Continuation-in-part of application No. 17/275,553.

(30) Foreign Application Priority Data
Sep. 13, 2018 (IN) 201841034585

(51) Int. Cl. C12P 21/02; C07K 14/605

(52) U.S. Cl. CPC C12P 21/02; C07K 14/605

(57) ABSTRACT

The present invention provided the techniques and recipes for enhancing recombinant peptide production in microorganism like E. coli, Saccharomyces cerevisiae, Pichia pastoris and Bacillus subtilis. The designs of fusion protein with a polypeptide and high cell density fermentation process to over express the peptides are given. Methods for separation of polypeptides from fusion protein and methods for isolation and purification of peptides are mentioned. This invention also provides an uncomplicated and unique purification processes for manufacturing of Teriparatide, Liraglutide precursor and Semaglutide precursor with purities of >98%.

### FIELD OF INVENTION

The present invention generally relates to compositions and techniques for improving recombinant peptide production in a microorganism. The recombinant peptides use fusion tags for stable and enhancing peptide production.

### BACKGROUND OF THE INVENTION

Great interest in therapeutic biotechnology is research on naturally occurring proteins and peptides, but obtaining them from natural sources is very difficult because of natural abundance.

So, the research has been focused on recombinant protein production in micro organisms by genetic engineering process to make a sufficient production.

Microorganisms like E. coli, P. pastoris and S. cerevisiae are the preferred hosts for recombinant protein expression. Proteins are often translated poorly or fold improperly from expression constructs, resulting in poor protein expression, solubility and ultimately low yield.

There is thus an ongoing and unmet need for improved compositions and methods for improving recombinant protein production.

Small synthetic peptides are found to be very important pharmaceutical tool and they are now being used for the treatment of various diseases and disorders. They are generally small protein-like chains designed to mimic a naturally existing peptide. Their altered chemical structure is designed advantageously to adjust the molecular properties such as stability or biological activity.

These small molecules can be selectively used as human GLP-1 analogue, parathyroid hormone analogue, agonist of guanylate cyclase-C etc. Some important examples of these small synthetic peptides are Teriparatide, Semaglutide and Liraglutide etc.

These small peptides are generally chemically synthesized. Most preferred method for the preparation of these peptides is solid-phase synthesis method in which an amino-protected amino acid is bound to a solid phase material (most commonly, low cross-linked polystyrene beads) forming a covalent bond between the carbonyl group and the resin. Then the amino group is deprotected and reacted with the carbonyl group of the next amino-protected amino acid and same way desired length of peptide is obtained.

Obtaining peptides by solid-phase synthesis method is very tedious, expensive and time consuming process and great expertise is required for this. In this method one residue is added at a time and after each residue change of chemical is required after each coupling step. Problem of repetitive coupling occurs as the length of peptide increases. Purification of synthesized polypeptide from the mixture of truncated and aberrant peptides is also very troublesome.

To overcome these problems in solid-phase synthesis method and meeting the increasing demand of these synthetic polypeptides various attempts has been made for the production of these peptides by expressing these peptides in microbial systems e.g. Bacteria and yeast, and subsequent recovery and purification of these peptides.

Liraglutide is produced in Saccharomyces cerevisiae using the recombinant DNA technology. Studies have proven that the E. coli derived liraglutide peptide production is preferred due to its considerable efficacy, economic production, and ease of process modification, optimization, and purification.

The expression of ubiquitin fusion protein or peptide in E. coli and the subsequent purification was first described by Monia et al, J. Biol. Chem. 264, 4093-4103 (Mar. 5, 1989) for production of carboxyl extension proteins (CEP). The carboxyl extension proteins (CEP) are naturally occurring proteins of 12 to 80 amino acids in length found in various eukaryotic organisms.

Ecker et al., J. Biol. Chem. 264, 7715-7719 (May 5, 1989) discloses the expression of cloned eukaryotic genes in microorganisms to allow for the isolation of large quantities of naturally occurring protein products which are present in only trace amounts from natural sources.

Butt et al., Proc. Natl. Acad. Sci. 86, 2540-2544 (April 1989) discloses an expression system for cloning ubiquitin-fusion proteins using E. coli. This further discloses that fusion of ubiquitin by its carboxyl terminal end to the N-terminus of these proteins increases the yield of unstable or poorly expressed proteins.

In 1986, Bachmair et al, Science 234, 179-186 (1986) suggests that ubiquitin may be helpful in preparing [beta]-galactosidase fusion proteins having any N-terminal amino acid when expressed in both bacteria and yeast.

PCT Publication No. WO2017/021819 A1 discloses the expression of peptides in E. coli using ubiquitin fusion tag which may also contain an affinity tag or linker or combination of affinity tag and linker. The affinity tag can be selected from Polyarginine-tag (Arg-tag), Polyhistidine-tag (His-tag), S-tag, SBP-tag (streptavidin-binding peptide), Maltose binding protein, Chitin binding domain (CBD) and linker is a peptide chain of acidic amino acids or basic amino acid, wherein chain length is of 1-10 acidic or basic amino acids. This document reports highest accumulation of a recombinant protein in E. coli up to 50% of the total cellular protein, i.e 50% specific yield.

PCT Publication No. WO2018/020417 also discloses the production of peptides in E. coli using ubiquitin fusion tag with affinity tag or SI tag or combination of affinity tag and SI tag in the expression vector. This document also reports the similar yields of a recombinant protein in E. coli as mentioned in Publication No. WO2017/021819 A1.

All reported prior art documents have one or other limitations so there is a need of modified process to overcome limitations of prior arts and produce greater amount of recombinant peptides in prokaryotic system and recover highly purified peptides as a final product.

### OBJECTIVE OF THE INVENTION

The main objective of the present invention is to provide a unique gene construct for the production of recombinant peptides in prokaryotic expression systems.

Another objective of the present invention is to maximize the production of recombinant peptides by high cell density fermentation.

In yet another objective of the present invention is to provide isolation and purification of recombinant peptides which involves enzymatic cleavage of the fusion protein using protease enzymes like enterokinase and ubiquitin hydrolase which generates the peptide of interest and purification of peptides by ion exchange and reverse phase chromatography to obtain highest purity.

### SUMMARY OF INVENTION

The first embodiment of the present invention relates to a synthetic oligonucleotide sequence encoding the peptides with STU/STE fusion tag consisting of a protease recognition sites.

The second embodiment of the present invention relates to a process for the preparation of peptides using recombinant microorganism which involves:
a) sequence encoding the peptides with STU/STE fusion tag consisting of a protease recognition site,
b) expression and isolation of fusion protein, and
c) fusion protein cleavage, isolation and purification of peptide

The third embodiment of the present invention relates to the process which comprises production of recombinant peptides in microorganism like E. coli, Saccharomyces cerevisiae, Pichia pastoris and Bacillus subtilis.

The fourth embodiment of the present invention relates to a process which comprises production of recombinant peptides in E. coli with HCDM3 medium and optimized process parameters during fermentation.

The fifth embodiment of the present invention relates to extraction of expressed fusion protein from recombinant microorganism by cell lysis and filtration.

### BRIEF DESCRIPTION OF DRAWINGS

FIG. 1 Shows amino acid sequence of STU solubility tag followed peptide sequence, (Three different STU tags were tried for protein expression and all the three has shown similar expression titers)

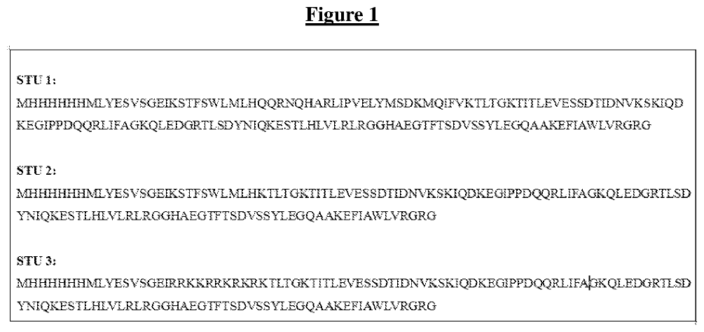

FIG. 2 Shows amino acid sequence of STE solubility tag followed peptide sequence, (Three different STE tags were tried for protein expression and all the three has shown similar expression titers)

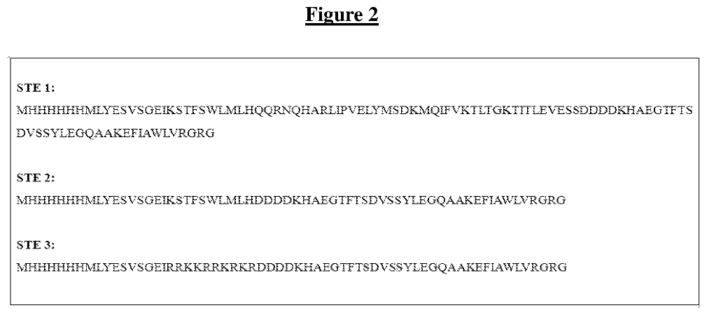

FIG. 3 Shows expression cassette comprising solubility tag with peptide sequence,

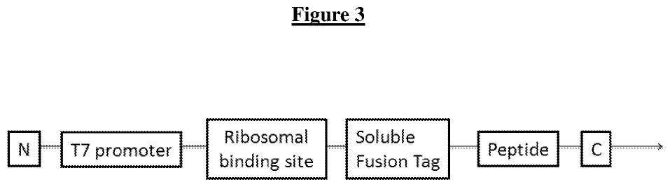

FIG. 4 Shows highest protein expression after 6 h of induction, and

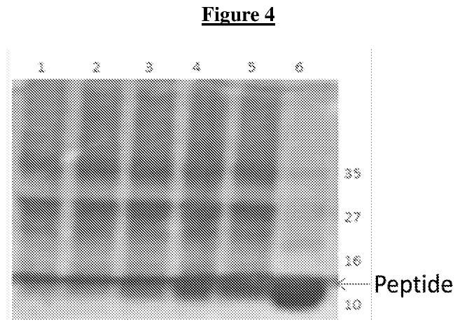

FIG. 5 Shows the flow chart of final purification process.

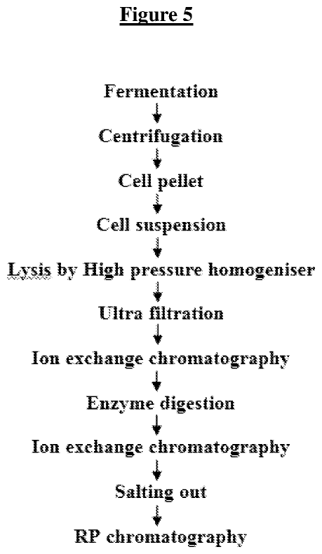

### DETAILED DESCRIPTION OF INVENTION

In describing the embodiments of the invention, specific terminology is resorted for the sake of clarity. However, it is not intended that the invention be limited to the specific terms so selected and it is to be understood that each specific term includes all technical equivalents that operate in a similar manner to accomplish a similar purpose.

Embodiments herein and the various features and advantageous details thereof are explained more fully with reference to the non-limiting embodiments that are illustrated in the accompanying figures and detailed in the following description. The examples used herein are intended merely to facilitate an understanding of ways in which the embodiments may be practiced and to further enable those skilled in the art to practice the embodiments herein. Accordingly, the examples should not be construed as limiting the scope of the embodiments herein.

The present invention is related to a novel process for production of recombinant peptides in prokaryotic expression system. In present invention all the natural peptides are expressed as a fusion protein and then the fusion tag is removed by an enzymatic digestion process. According to the present invention, natural peptides can be produced more efficiently when compared with prior art processes.

Present invention also provides an optimized high yielding fermentation media for the expression of natural peptides like Teriparatide, Liraglutide and Semaglutide precursor in microorganism.

This invention also provides a unique purification process for Teriparatide, Liraglutide and Semaglutide precursor by using protein precipitation, ion exchange chromatography and RP-HPLC methods. The process yields more than 95% purity of the peptides.

The microorganism used herein may be selected from B. subtilis, E. coli, P. pastoris and S. cerevisiae.

The fusion protein of the present invention is soluble form which contains 90-130 amino acids. With the fusion tag used in the present invention the unfolding of protein does not take place and hence refolding is not required. In view of this, affinity chromatography is avoided for purification; instead the purification can be carried out using simple purification techniques like precipitations, ultra filtrations and conventional ion exchange chromatography techniques. This is a significant change over the prior-art which reduces the overall cost of production.

The isolation of fusion protein comprises usage of 100 to 1000 kDa of cross flow filters for clarification of cell lysate or fermentation broth and usage of 1-8 molar urea in lysate or cell suspension or in broth.

The cleavage process of fusion protein comprises the enzyme and substrate ratios of 1:5 to 1:100, wherein the enzymes are enterokinase, ubiquitin hydrolase and the substrates are STE/STU fusion proteins

The cleavage process of fusion protein is carried out at a temperature in the range from 15-35° C. at pH in the range from 5-9, wherein the process carried out by ultra filtration (100 to 1000 kDa) and cell lysate or broth suspension using buffer containing 1 to 8M urea. The simultaneous steps of cleavage and precipitation to isolate peptides at temperature in the range from 0-10° C. at pH in the range from 4-7.

The representative example Liraglutide, Teriparatide and Semaglutide used herein are intended merely to facilitate an understanding of ways in which the embodiments of the invention may be practiced and to further enable those skills in the art to practice the embodiments herein. Accordingly, this example should not be construed as limiting the scope of the embodiments herein.

According to the present invention, the purity of the peptides will be in range of 95% to 99% when measured by using RP-HPLC (with the purities of Teriparatide>98%, Liraglutide precursor >95% and Semaglutide precursor >95%.

Based on the microorganism used for expression, the location of the fusion protein expression and the protein isolation process will change. Cleavage of fusion protein is same irrespective of organism and the peptide. It only differs in conditions of cleavage and ratio of enzyme and substrate.

Further, the isolation and purification of the final peptide will change with respect to the end peptide.

### Stage 1: Clone Development

### 1. Cloning and Over Expression of Peptides in Microorganism Like Bacteria and Yeast

For the over expression of peptides in the selected microorganism, following two cloning strategies were employed with fusion tags either in an expression vector or chromosomal insertions.

1. STU solubility tag followed by peptide (STU)

2. STE solubility tag followed by peptide (STE)

2. Cloning and Expression of Peptide with Solubility Tag STU/STE in E. coli.

a. Cloning of Synthetic Gene Construct (Peptide) with STU/STE Fusion Tag
The synthetic gene construct of peptide with STU/STE fusion tag was obtained as a synthetic construct cloned in pUC57 cloning vector. The synthetic construct was transformed into E. coli DH5α and the desired fragment was isolated by restriction digestion of the plasmid DNA with the restriction enzymes Nde1, Xho1, BamHI and SacI.

b. Sub-Cloning of Peptide with STU/STE Fusion Tag into Expression Vector
The expression vector (pET24/pET28) was digested with Nde1, Xho1, BamHI and SacI for cohesive end ligation. The digested plasmid and the above isolated STU-peptide/STE-peptide insert were ligated in a molar ratio of 3:1. The ligated product (pET24/28: STU-peptide/STE-peptide) was transformed into E. coli DH5α by heat shock method and transformants were selected by antibiotic selection marker (Kanamycin (50 μg/ml)).

c. Characterization of Recombinant Clone of E. coli DH5a Containing Recombinant Expression Vector (pET24/28STU-Peptide/STE-Peptide)
Positive colonies were initially characterized by PCR amplification (colony PCR method) using T7 primers and finally characterized by restriction enzyme digestion (Nde1, Xho1, BamHI and SacI).

d. Expression Studies of Peptide in E. coli Expression Hosts

For expression studies, the characterized recombinant expression plasmid (pET24/28 STU-peptide/STE-peptide) was transformed into E. coli expression host BL21 (DE3) by heat shock method and the transformants were selected using kanamycin as a selection marker.

To check expression of peptide with STU/STE tag, one colony from each plate is inoculated into LB medium and grown at 37° C. When the culture reached to mid-log phase (O.D of 0.6 to 0.7), the cells were induced with 1 mM IPTG. After induction, 1 ml of sample was collected from both un-induced and induced cultures at 0, 1, 2 and 3 hrs to check the expression on SDS gel.

Expression of the peptide with STU/STE tag was observed in all the constructs which were designed. Each one of them was designed keeping in mind of the different methods employed for the removal of solubility tag. The STU/STE tag can be cleaved by the addition of ubiquitin hydrolase/Enterokinase enzyme respectively.

When the cleavage efficacy of enterokinase and ubiquitin hydrolase was tested, STU and STE fusion peptides, both the tags were found to be the best choice. The cleavage efficiency of enterokinase is high comparing with ubiquitin hydrolase.

### Stage 2: Fermentation Process Development

Comparison Recombinant Protein Expression and Titers with Different Fermentation Medium:
A total of seven different media compositions and process parameters were tested for protein expression and titers. Media 7 (HCDM 3) was found to be the best media for E. coli growth and fusion protein expression

### Media 1: Luria Bertani Broth

### Composition:

| Component | g/L |
|---|---|
| Tryptone | 10.0 |
| Yeast extract | 5.0 |
| Sodium chloride | 5.0 |

### OD₆₀₀: 4-6

Protein titer: 100-150 mg/L
Media 2: Terrific Broth with Glucose

### Composition:

| Component | g/L |
|---|---|
| Tryptone | 12 |
| Yeast extract | 24 |
| Glucose | 20 |
| Dipotassium hydrogen phosphate | 9.4 |
| Potassium dihydrogen phosphate | 2.2 |

### OD₆₀₀: 12-15

Protein titer: 250-350 mg/L

### LB and Terrific Broth Media:

LB, Terrific broth-glucose and Terrific broth-glycerol were tested for cell growth and protein expression. The growth and protein expressions are observed less in LB, Terrific broth-glucose compared to Terrific broth glycerol. Since the growth and protein expressions are better in Terrific broth-glycerol, feeding was applied to increase the expression levels and cell growth.
Media 3: Terrific Broth with Glycerol

### Composition:

| Component | g/L |
|---|---|
| Yeast extract | 24 |
| Glucose Glycerol | 5 |
| Dipotassium hydrogen phosphate | 9.4 |
| Potassium dihydrogen phosphate | 2.2 |

### OD₆₀₀: 15-20

Protein titer: 400-500 mg/L
Media 4: Terrific Broth with Feed

### Composition:

| Component | g/L |
|---|---|
| Tryptone | 12 |
| Yeast extract | 24 |
| Glycerol | 5 |
| Dipotassium hydrogen phosphate | 9.4 |
| Potassium dihydrogen phosphate | 2.2 |

### Feed Composition:

| | (g/L) | |
|---|---|---|
| Feed A | | |
| Glucose | 500 | |
| Thiamine | 1 | ml |
| Trace elements | 2 | ml |
| Feed B | | |
| Tryptone | 12 | g |
| Yeast extract | 24 | g |
| Trace element solution | | |

| | |
|---|---|
| FeSO₄7H₂O | 10 |
| ZnSO₄7H₂O | 2.25 |
| CuSO₄5H₂O | 1.0 |
| MnSO₄H₂O | 0.5 |
| Na₂B₄O₇ 10 H₂O | 0.23 |
| CaCl₂ | 2.0 |
| (NH₄) ₆Mo₇O₂4H₂O | 0.1 |

### OD₆₀₀: 25-30

Protein titer: 1.0-1.5 g/L
Terrific broth with feed: In order to increase the cell density the tank medium was given glucose at basal concentration and further fed with concentrated glucose and yeast extract to meet the C:N ratio requirement. There was an increase in cell growth (25-30 O.D) and protein expression (1-1.5 g/L) with feed addition.

### Media 5: HCDM 1

### Composition:

### Tank Media:

| Component | g/L |
|---|---|
| Potassium dihydrogen phosphate | 2.2 |
| Ammonium sulphate | 4.5 |
| Citric acid | 1.0 |
| Glucose | 20 |
| MgSO₄ | 130 ml |
| | (70 mg/ml |
| | concentration) |
| Cacl₂ | 5 ml |
| | (80 mg/ml |
| | concentration) |
| Trace metal solution | 10 ml |
| Thiamine Hcl | 2 ml |
| | (100 mg/ml |
| | concentration) |

### Trace Metal Solution:

| Component | mg/10 ml | Volume (ml) |
|---|---|---|
| EDTA | 500 | 5 |
| FeSO₄ | 500 | 10 |
| ZnSO₄ | 500 | 2 |
| MnSO₄ | 500 | 2 |
| CoCl₂ | 500 | 0.2 |
| CuSO₄ | 500 | 0.1 |
| Na₂MoO₄ | 500 | 0.2 |
| H₃BO₃ | 500 | 0.1 |

### Feed Medium Composition:

| Component | g/L | |
|---|---|---|
| Glucose | 180 | gm |
| Tryptone | 50 | gm |
| MgSO₄ | 57.5 ml (70 mg/ml concentration) | |
| Cacl₂ | 25 ml (80 mg/ml concentration) | |
| Trace metal solution | 6 | ml |
| Thiamine Hcl | 0.2 ml (100 mg/ml concentration) | |
| FeSO₄ | 500 | mg |
| Trace metal solutions | 50 | ml |

All the components were added and made up to volume of 1 liter.

### OD₆₀₀: 40-50

Protein titer: 2.0-2.5 g/L

### Media 6: HCDM 2

### Composition:

| Media Component | g/L |
|---|---|
| Tryptone | 20 |
| Yeast extract | 10 |
| Sodium chloride | 5 |
| Dextrose | 5 |
| Potassium chloride | 1 |
| Magnesium sulphate | 2 |
| Magnesium chloride | 2 |

### Feed Medium: 2 L

| Media Component | g/L |
|---|---|
| Yeast Extract | 150 |
| Dextrose Anhydrous | 500 |

### OD₆₀₀: 65-70

Protein titer: 3.0-4.0 g/L

### Media 7: HCDM 3

### Composition:

### Production Media:

| Media Component | g/L |
|---|---|
| Tryptone | 20 |
| Yeast extract | 10 |
| Sodium chloride | 5 |
| Dextrose | 5 |
| Potassium chloride | 1 |
| Magnesium sulphate | 2 |
| Magnesium chloride | 2 |

Feed Medium: 2 L (for 7.2 liters)

| Media Component | g/L |
|---|---|
| Yeast Extract | 150 |
| Lactose | 250 |
| MgSO₄ | 1.5 |
| (NH₄)₂ SO₄ | 1.5 |

### OD₆₀₀: 80-90

Protein titer: 5-6 g/L

### Results:

### High Cell Density Media:

Three different fermentation Media were used to increase the cell density and protein expression, OD₆₀₀ 40-50, 50-60 and 70-80 were achieved with HCDM 1, HCDM 2 and HCDM 3 respectively, whereas the protein titers are 2-2.5 g/L, 3-4 g/L and 5-6 g/L.
HCDM 3 medium has shown better results than HCDM 1 and HCDM 2.

### Conclusion:

Based on the above experimental data, HCDM 3 media and below process parameters were freezed as optimum conditions for peptide production.
Seed media: LB broth
Seed age: 7 to 10 hours
OD600: 1.7 to 2.5 (mid log phase)
Fermentation process:

### Media: HCDM 3

Batch phase duration: 6-9 hours
Fed batch duration: 7-10 hours

### Harvest OD₆₀₀: 80 to 90

Production media for 10 liters
Production media: 7.2 ltrs

| Media Component | g/L |
|---|---|
| Tryptone | 20 |
| Yeast extract | 10 |
| Sodium chloride | |
| Dextrose | 5 |
| Potassium chloride | 1 |
| Magnesium sulphate | 2 |
| Magnesium chloride | 2 |

### Feed Medium: 2 L

| Media Component | g/L |
|---|---|
| Yeast Extract | 150 |
| Lactose | 250 |
| MgSO₄ | 1.5 |
| (NH₄)₂ SO₄ | 1.5 |

### Stage 3: Purification Process Development

### 1. Isolation and Purification of Fusion Protein:

### Trial 1:

1. 10% cell suspension was prepared in 20 mM Sodium phosphate buffer, 8M urea pH 7.2 and passed through high pressure homogenizer at 800 bar pressure for cell lysis, repeated the passages till the suspension OD₆₀₀ decreases to <5.
2. 0.6μ Cross flow filtration was done for cell lysate, Fusion peptide was collected in filtrate and the retentate contained cell debris and other impurities. 90% of the fusion peptide was recovered in filtrate with purity of around 40-50%.
3. Filtrate was loaded on affinity chromatography (Ni-Sepharose) and eluted the fusion protein using 250 mM imidazole with a purity of >80%
4. The above purified fusion protein was applied for enzymatic cleavage to separation the peptide from fusion tag.

### Trial 2:

1. 10% cell suspension was prepared in 20 mM Tris buffer, 6M urea pH 8.5 and passed through high pressure homogenizer at 800 bar pressure for cell lysis, repeated the passages till the suspension OD₆₀₀ decreases to <5.
2. 0.2μ Cross flow filtration was applied for cell lysate, Fusion peptide was collected in filtrate and the retentate contained cell debris and other impurities. 90% of the fusion peptide was recovered in filtrate with purity of around 50-60%.
3. Filtrate was loaded on affinity chromatography (Ni-Sepharose) and eluted the fusion protein using 250 mM imidazole with a purity of >80%
4. The above purified fusion protein was applied for enzymatic cleavage to separation the peptide from fusion tag.

### Trial 3:

1. 10% cell suspension was prepared in 20 mM Tris buffer pH 8.5 and passed through high pressure homogenizer at 800 bar pressure for cell lysis, repeated the passages till the suspension OD₆₀₀ decreases to <5.
2. 2M urea was added to the cell lysate, dissolved and then passed through 750 kDa Cross flow filtration, Fusion peptide was collected in filtrate and the retentate contained cell debris and other impurities. 90% of the fusion peptide was recovered in filtrate with purity of around 90%.
3. The above purified fusion protein was applied for enzymatic cleavage to separate the peptide from fusion tag.

### Conclusion:

Based on the above three trial run results, in trial 3 got the best recovery and purity

### 2. Fusion Protein Digestion

Digestion with Light Chine Enterokinase:
The enzyme enterokinase cuts STE fusion protein exactly at the peptide part to release peptide. Enterokinase enzyme was added to the purified fusion protein at pH 8.0, 2 mM CaCl₂ and incubated at 35° C. under mild stirring and the percentage of cleavage was tested by RP-HPLC. ≥90% cleavage was recorded in 5 hours duration at 30° C. under mild stirring.
Digestion with Ubiquitin Hydrolase:
The enzyme ubiquitin hydrolase cuts STU fusion protein exactly at the peptide part to release peptide. Ubiquitin hydrolase enzyme was added to purified fusion protein at pH 8.0 and incubated at 25° C. under mild stirring and the percentage of cleavage was tested by RP-HPLC. ≥90% cleavage was recorded in 3 hours duration at 30° C. under mild stirring.
The enzyme and substrate ratios were tried for 1:5 to 1:100 for both the enzymes enterokinase and yeast ubiquitin hydrolase. In both the cases 1:15 was found to be optimum.

### 3. Isolation and Purification of Peptide:

In both the strategies (STU and STE) the cloning, fermentation, purification and digestion of fusion protein are similar to all natural peptides.

### EXAMPLES

### Example 1: Isolation and Purification of Liraglutide Precursor

1. 3.0M sodium chloride was added to the digestion mixture and the pH of the sample was reduced to 7.0 using diluted HCl.
2. Centrifuged the sample to remove separated fusion tag and fusion protein from the separated peptide. Fusion tag and fusion protein were come in pellet and 90% of the peptide was in supernatant.
3. Centrifuged supernatant was further adjusted the pH to 4.0 using diluted HCl and collected the pellet for peptide.
4. Peptide pellet was dissolved in 20 mM Tris pH 8.5 buffer and tested for purity by RP-HPLC and it was 90-95% pure. It was used for liraglutide preparation.

### Example 2: Isolation and Purification of and Purification of Teriparatide

1. 3.5M sodium chloride was added to the digestion mixture and the pH of the sample was reduced to 7.0 using diluted acetic acid.
2. Centrifuged the sample to remove separated fusion tag and fusion protein from the separated peptide. Fusion tag and fusion protein were come in pellet and 90% of the peptide was in supernatant.
3. Centrifuged supernatant was further adjusted the pH to 5.5 using diluted acetic acid and collected the pellet for peptide.
4. Peptide pellet was dissolved in 10 mM Sodium acetate pH 4.0 buffer and tested for purity by RP-HPLC and it was 90-95% pure. It was further purified for Teriparatide API preparation.

### Example 3: Isolation and Purification of and Purification of Semaglutide Precursor

1. 3.5M sodium chloride was added to the digestion mixture and the pH of the sample was reduced to 7.5 using diluted HCl.
2. Centrifuged the sample to remove separated fusion tag and fusion protein from the separated peptide. Fusion tag and fusion protein were come in pellet and 90% of the peptide was in supernatant.
3. Centrifuged supernatant was further adjusted the pH to 4.5 using diluted HCl and collected the pellet for peptide.
4. Peptide pellet was dissolved in 10 mM Tris pH 8.5 buffer and tested for purity by RP-HPLC and it was 90-95% pure. It was further used for semaglutide preparation.

1: A synthetic oligonucleotide sequence encoding the peptides with STU/STE fusion tag consisting of a protease recognition sites, wherein the STU and STE sequence code is as given below:
STU1:MHHHHHHMLYESVSGEIKSTFSWLMLHQQRNQHARLIPVELYMSDK
MQIFVKTLTGKTITLEVESSDTIDNVKSKIQDKEGIPPDQQRLIFAGKQLE
DGRTLSDYNIQKESTLHLVLRLRGGHAEGTFTSDVSSYLEGQAAKEFIAWL
VRGRG
STU2:MHHHHHHMLYESVSGEIKSTFSWLMLHKTLTGKTITLEVESSDTID
NVKSKIQDKEGIPPDQQRLIFAGKQLEDGRTLSDYNIQKESTLHLVLRLRG
GHAEGTFTSDVSSYLEGQAAKEFIAWLVRGRG
STU3:MHHHHHHMLYESVSGEIRRKKRRKRKRKTLTGKTITLEVESSDTID
NVKSKIQDKEGIPPDQQRLIFAGKQLEDGRTLSDYNIQKESTLHLVLRLRG
GHAEGTFTSDVSSYLEGQAAKEFIAWLVRGRG
STE1:MHHHHHHMLYESVSGEIKSTFSWLMLHQQRNQHARLIPVELYMSDK
MQIFVKTLTGKTITLEVESSDDDDKHAEGTFTSDVSSYLEGQAAKEFIAWL
VRGRG
STE2:MHHHHHHMLYESVSGEIKSTFSWLMLHDDDDKHAEGTFTSDVSSYL
EGQAAKEFIAWLVRGRG
STE3:MHHHHHHMLYESVSGEIRRKKRRKRKRDDDDKHAEGTFTSDVSSYL
EGQAAKEFIAWLVRGRG

2: The peptides as referred in claim 1, wherein the peptides relate to Teriparatide, Liraglutide and Semaglutide precursor.

3: The peptides as referred in claim 1, wherein the fusion tag is a soluble form which contains 90 to 130 nucleic acids, wherein the unfolding of protein does not take place and hence refolding is not required.

4: A process for preparation of peptides using recombinant microorganism which involves
a) cloning of gene sequence encoding the peptides with STU/STE fusion tag consists of a protease recognition site,
b) expression and isolation of fusion protein, and
c) fusion protein cleavage, isolation and purification of peptide.

5. (canceled)

6: The process as claimed in claim 4, wherein the process comprises expressing the peptides as a fusion protein and then the fusion tag is removed by an enzymatic digestion process.

7: The process as claimed in claim 4 wherein the process comprises production of recombinant peptides of the peptides in microorganism which is E. coli, Saccharomyces cerevisiae, Pichia pastoris and Bacillus subtilis.

8: The process as claimed in claim 4, wherein the process comprises production of recombinant peptides in E. coli with HCDM3 medium and optimized process parameters during fermentation.

9: The process as claimed in claim 4, wherein the isolation of fusion protein comprises usage of 100 to 1000 kDa of cross flow filters for clarification of cell lysate or fermentation broth and usage of 1-8 molar urea in lysate or cell suspension or in broth.

10: The process as claimed in claim 4, wherein the cleavage process of fusion protein comprises the enzyme and substrate ratios of 1:5 to 1:100, wherein the enzymes are enterokinase, ubiquitin hydrolase and the substrates are STE/STU fusion proteins.

11: The process as claimed in claim 4, wherein the process comprises unique purification process for Teriparatide, Liraglutide and Semaglutide precursor by using protein precipitation, ion exchange chromatography and RP-HPLC methods.

12. (canceled)

13: The process as claimed in claim 4, wherein the cleavage process of fusion protein is carried out at a temperature in the range from 15-35° C.

14. (canceled)

15: The process as claimed in claim 4, wherein the process carried out by ultra filtration (100 to 1000 kDa) and cell lysate or broth suspension using buffer contains 1 to 8M urea.

16. (canceled)

17. (canceled)
